(19) United States
(12) Patent Application Publication
(10) Pub. No.: US 2023/0355854 A1
(43) Pub. Date: Nov. 9, 2023

(54) BLOOD TREATMENT SYSTEM AND METHODS OF CONTROLLING SAME

(71) Applicant: QIDNI LABS INC., Buffalo, NY (US)

(72) Inventors: Morteza AHMADI, Buffalo, NY (US); Clarence GRAANSMA, Buffalo, NY (US)

(73) Assignee: QIDNI LABS INC., Buffalo, NY (US)

(21) Appl. No.: 17/906,543

(22) PCT Filed: Mar. 16, 2021
(86) PCT No.: PCT/US2021/022514

§ 371 Date: Sep. 16, 2022

Related U.S. Application Data
(60) Provisional application No. 62/990,206, filed on Mar. 16, 2020.

(51) Int. Cl. A61M 1/36; A61M 1/16

(52) U.S. Cl. CPC A61M 1/3641; A61M 1/1633; A61M 2205/3341

(57) ABSTRACT

A blood treatment system and method controlling same are provided. The system comprises a blood pump for urging blood from an arterial or venous interface through a blood flow path; a dialyser in fluid communication with said blood flow path for ultrafiltering the blood to remove fluid therefrom; a fluid removal pump in fluid communication with said dialyser for urging ultrafiltered fluid away from said dialyser; a controller in signal communication with said blood pump; and a reversing valve for selectively reversing direction of blood flow in at least a portion of the blood flow path under signal control of said controller. The blood pump is selectively activatable under signal control of the controller.

### CROSS REFERENCE TO RELATED APPLICATION AND CLAIM OF PRIORITY

The present application claims priority to U.S. Provisional Pat. Application No. 62/990,206 filed on Mar. 16, 2020, the entire contents of which are hereby incorporated by reference.

### BACKGROUND

### Technical Field

The disclosure related generally to blood treatment, and more particularly to systems and methods for ultrafiltration of blood.

### Background

Renal failure may require hemodialysis for extended periods of time. Conventional hemodialysis regimes may utilize 3 four-hour treatments per week which is a common treatment regimen for end-stage renal failure. This regimen has 2 intervals between dialysis treatments of about 44 hours and 1 interval between dialysis treatments of about 68 hours each week. Many patients dialysing on this regimen do not well tolerate the fluid and sodium accumulation that occurs between the treatments, which may cause them to suffer from poor blood pressure control and the other complications. Complication may be severe during the 68-hour interval and during the hemodialysis treatment immediately following the 68-hour interval. One solution that has been used to deal with this problem is to schedule 4 hemodialysis treatments per week instead of 3 treatments per week so as to eliminate the 68-hour interval for the patients that are least able to tolerate the conventional regimen. This additional treatment each week may increase logistical and operating costs for healthcare providers, reimbursement and insurance agencies. Patients may also resist attending this fourth scheduled treatment, especially when it is on the day immediately following another treatment as the patients do not yet feel the effects of the impending fluid overload yet and so they may not show up for a scheduled treatment.

### SUMMARY

The disclosure herein is directed to blood treatment systems and methods for controlling the blood treatment systems, which comprise a ultrafiltration dialyzer(s) to be used for a low flow, extended length treatment modality. The system and methods may allow prolonged treatment which may minimize fluid and sodium accumulation in a patient. The blood treatment system and methods include automation of functions that are normally performed manually by the patient in other existing home hemodialysis modalities. Incorporated control and monitoring systems may also incorporate certain safety features found in larger renal therapy systems.

In an aspect a blood treatment system is provided. The system comprises a blood pump for urging blood from an arterial or venous interface through a blood flow path; a dialyser in fluid communication with the blood flow path for ultrafiltering the blood to remove fluid therefrom; a fluid removal pump in fluid communication with the dialyser for urging ultrafiltered fluid away from the dialyser; a controller in signal communication with the blood pump; and a reversing valve for selectively reversing direction of blood flow in at least a portion of the blood flow path under signal control of the controller, wherein the blood pump is selectively activatable under signal control of the controller.

In an embodiment, the dialyser is a first dialyser and the system further comprises a second dialyser.

In an embodiment, the system comprises at least one flow control element for directing the blood flow path through a selected one of the first dialyser or the second dialyser under signal control of the controller. In an embodiment, the at least one flow control element includes a clamp or a valve.

In an embodiment, the portion of the blood flow path comprises the arterial and the venous interface.

In an embodiment, the reversing valve is positioned upstream of an inlet of the blood pump for selectively flowing blood from one of the arterial or venous interface to the inlet of the blood pump without reversing the blood pump.

In an embodiment, the system comprises a prime fluid reservoir. In another embodiment, the system further comprises a plurality of other valves, each under signal control of the controller to effect automated priming of the system by circulating prime fluid urged by the blood pump from the prime fluid reservoir along selected fluid flow paths. In another embodiment, the plurality of other valves includes a plurality of minimal dead space valves. In another embodiment, the plurality of other valves includes a recirculating valve for controlling return of blood. In another embodiment, the selected fluid flow paths includes a fluid flow path connecting the prime fluid reservoir and the first dialyser. In another embodiment, the selected fluid flow paths includes a fluid flow path connecting the prime fluid reservoir and the second dialyser. In another embodiment, the selected fluid flow paths includes a fluid flow path connecting the prime fluid reservoir and the arterial interface. In another embodiment, the selected fluid flow paths includes a fluid flow path connecting the prime fluid reservoir and the venous interface. In another embodiment, the controller is configured to control the other valves to remove the first dialyser from the blood flow path to facilitate replacement of the first dialyser. In another embodiment, the controller is configured to control the other valves to remove the second dialyser from the blood flow path to facilitate replacement of the second dialyser.

In an embodiment, the system comprises an air removal filter for removing air bubbles from the blood. In another embodiment, the air removal filter includes an orientation sensor for detecting an orientation of the air removal filter relative to ground. In another embodiment, the system comprises a motor for rotating the air removal filter about at least two axis of rotation.

In an embodiment, the system comprises an air detector for detecting air in the blood. In another embodiment, the controller implements logic for disabling the blood pump in response to detection of air in the blood by the air detector.

In an embodiment, the system comprises an anticoagulant source in fluid communication with the blood flow path, for adding anticoagulant to the blood flow path.

In an embodiment, the system comprises a pressure sensor for measuring pressure of the blood proximate the arterial or venous interface. In another embodiment, the controller implements logic for disabling the blood pump in response to detection of the pressure of the blood by the pressure sensor in excess of a pre-defined limit.

In an embodiment, the system comprises a blood sensor for sensing blood in the ultrafiltered fluid. In another embodiment, the controller implements logic for disabling the blood pump in response to detection of blood by the blood sensor.

In an embodiment, the fluid removal pump is reversible under signal control of the controller, to urge ultrafiltered fluid into the dialyser to clear fouling from a membrane of the dialyser.

In an embodiment, the controller is configured for reversing the fluid removal pump in response to determining a transmembrane pressure of the dialyser.

In an embodiment, the controller is configured for controlling a pump rate of the blood pump based on a measured fluid removal rate.

In an embodiment, the system comprising non-transitory memory for storing data received at the controller. In another embodiment, the data includes sensor data.

In an embodiment, the system is portable by a patient undergoing blood treatment.

In an embodiment, the system is wearable by a patient undergoing blood treatment.

Embodiments may include combinations of the above features.

In an aspect a method for controlling a blood treatment system is provided. The method comprises: causing a blood pump to flow blood through a flow path from one of an arterial or venous interface to a dialyser and to the other of the arterial or venous interface; and causing a reversing valve to reverse the flow of blood through a portion of the flow path.

In an embodiment, causing the reversing valve to reverse the flow of blood through the portion of the flow path comprises causing blood to reverse flow between the arterial and venous interface.

In an embodiment, the reversing valve is positioned upstream of an inlet of the blood pump, and the method comprises selectively flowing blood from one of the arterial or venous interface to the inlet of the blood pump without reversing the blood pump.

In an embodiment, the method comprises directing the blood flow path through a selected one of a first dialyser or a second dialyser.

In an embodiment, the method comprises causing a plurality of flow valves to prime a portion of the flow path by circulating prime fluid urged by the blood pump from a prime fluid reservoir along the portion of the flow path. In another embodiment, the method comprises recirculating the prime fluid through the portion of the flow path. In another embodiment, the portion of the flow path is isolated from a second dialyser. In another embodiment, the portion of the flow path includes the prime fluid reservoir and the arterial interface.

Embodiments may include combinations of the above features.

### BRIEF DESCRIPTION OF THE DRAWINGS

Reference is now made to the accompanying drawings, in which:

FIG. 1 shows an example blood treatment system in a forward mode of operation.

FIG. 2 shows the example system of FIG. 1 in a reverse mode of operation

FIG. 3 shows an example blood treatment system with one dialyzer in a forward mode of operation.

FIG. 4 shows the example blood treatment system of FIG. 3 in a reverse mode of operation.

FIG. 5 illustrates a schematic diagram of a system for controlling a blood treatment system.

### DETAILED DESCRIPTION

The disclosure herein describes systems and methods for blood treatment and more particularly to a portable and wearable ultrafiltration system to be used for a low flow, extended length treatment modality.

Most dialysis clinics are structured on a 7 day week with preference to reducing operations weekends, particularly with an attempt to remain closed or minimize operations on Sundays. This schedule structure generally meets the needs of clinic staff and patients in that it meshes well with the schedules of the rest of society. The majority of patients will be satisfactorily dialyzed with a standard treatment of about 4 hours repeated 3 times in each 7-day weekly cycle.

Using the standard 3 times per week 4-hour treatment in a 7-day rotating schedule means that there will be 2 intervals of 44 hours and 1 interval of 68 hours. It has been shown that some patients do not tolerate the longer 68-hour interdialytic interval very well and so have an increased risk of death or morbidity complications during or immediately following this longer interdialytic interval. This extended interdialytic interval could be eliminated by scheduling 7 treatments in each 14-day rotating cycle. This would require that patients dialyze on different days of the week in the first week of the schedule than in the second week of the schedule. This particular schedule is often used in home dialysis as there are no issues related to transportation, clinic staff scheduling or requiring a facility to be open for additional hours but is not easily implemented in clinics. Another more commonly used method to deal with this need is to schedule a fourth treatment in the clinic each week for patients that cannot tolerate the long interdialytic interval.

The clinical requirements for the 4th treatment are primarily to reduce the additional accumulated fluid volume and sodium that makes it difficult to maintain stable blood pressures near the end of a long interdialytic interval or during the dialysis treatment immediately following a long interdialytic interval. In most cases, the weekly requirements for toxin removal, buffer replacement, and control of other electrolytes are adequately met by the first three treatments each week. Clinical requirement may require additional removal of fluid and sodium which can be accomplished with ultrafiltration. In some examples, ultrafiltration without urea or other toxin removal may be better tolerated than hemodialysis. Ultrafiltration at lower rates over longer time periods may be better tolerated than ultrafiltration at higher rates over shorter time periods.

Blood flow rates for conventional in-center hemodialysis are usually maximized to increase the effective clearance of toxins in the allotted treatment time interval. Blood flow rates for hemodialysis generally range from 200 ml/min to 500 ml/min. In a wearable device utilizing such higher blood flow rates might result in a higher risk of access trauma, blood loss in case of inadvertent line separation, hemolysis, as well as more frequent alarms and increased risk of air embolism. It is therefore advantageous from a safety aspect to use lower blood flow rates in wearable blood treatment therapy methods. The primary technical limiting factor for the ultrafiltration rate is the blood flow rate, where the ultrafiltration rate should not exceed 20% of the blood flow rate. For an ultrafiltration treatment removing 10 liters of fluid, the minimum blood processed requirement would be 50 liters. If these 50 liters (50,000 ml) were to be processed over 68 hours (4,080 minutes), the absolute minimum blood flow rate would be 12.3 ml/min.

In some embodiments the system and methods described herein may provide a portable and wearable blood treatment system for fluid and sodium removal from a patient following conventional hemodialysis treatment for a duration of time, e.g. a 68-hour interval between conventional hemodialysis treatments, and then remove it from the patient at the end the duration. The system may automate various aspects of the required clinical setup and monitoring tasks so as to improve patient satisfaction and compliance as well as reduce training costs and clinical oversight costs borne by a health care provider. The system may also mitigate problems of clotting and fouling of blood circuit pathways that are common in any such device that is used for an extended time period. In particular, example functions that may be automated or simplified include: priming a fluid flow path, removing accumulated air, reversing the blood flow in the needles or catheter used for access, a restorative mode to remove fouling from ultrafiltration membrane (e.g. in a dialyser), replacement of excessively fouled or clotted ultrafiltration units (e.g. dialysers), returning the blood from the circuit to the patient, and emptying accumulated fluid from the device.

In some embodiments of the system and methods described, this disclosure provides a portable and wearable blood treatment system intended to be used to remove fluid from blood on a slow continuous basis. Such a device may be used to reduce the morbidity and mortality risk to hemodialysis patients during and immediately following long interdialytic intervals but may also be used for any purpose requiring fluid removal from the blood, such as for patients suffering from fluid accumulation related to congestive heart failure for instance.

A description of a plurality of operation modes of blood treatment system (100) follows with reference to the valve states summarized in Table 1.

TABLE 1

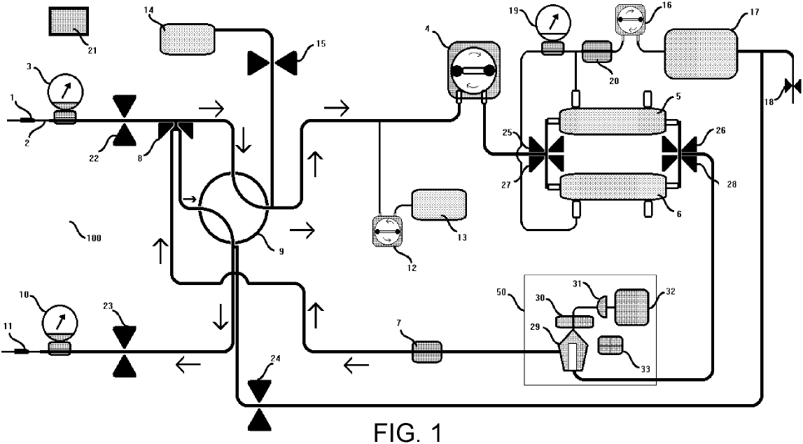

valves state mode table for valves illustrated in FIGS. 1-4 . Legend: O = Open; X= Closed; Rot = rotation; For = forward; Rev = reverse; D1 = first dialyzer; D2 = second dialyzer; Art = arterial interface; Ven = venous interface

| | | | | | | | | | | |
|---|---|---|---|---|---|---|---|---|---|---|
| Mode | V-Rot | V-Art (22) | V-Ven (23) | V-Rec (8) | V-Pri (15 ) | V-Uf (24) | V-D1-in (25) | V-D1-out (26) | V-D2-in (27) | V-D2-out (28) |
| Forward Operation D1 | For | O | O | X | X | X | O | O | X | X |
| Reverse Operation D1 | Rev | O | O | X | X | X | O | O | X | X |
| Forward Operation D2 | For | O | O | X | X | X | X | X | O | O |
| Reverse Operation D2 | Rev | O | O | X | X | X | X | X | O | O |
| Prime 1 - Intake - D1 | For | X | X | X | O | O | O | O | X | X |
| Prime 1 - Intake - D2 | For | X | X | X | O | O | X | X | O | O |
| Prime 2 - Circulate - D2 | For | X | X | O | O | X | X | X | O | O |
| Prime 2 - Circulate - D1 | For | X | X | O | O | X | O | O | X | X |
| Prime 3 - Flush Circuit - D1 | For | X | X | X | O | O | O | O | X | X |
| Prime 3 - Flush Circuit - D2 | For | X | X | X | O | O | X | X | O | O |
| Prime 3 - Flush Ven - D2 | For | X | O | X | O | X | X | X | O | O |
| Prime 3 - Flush Art - D1 | Rev | O | X | X | O | X | O | O | X | X |
| Return 1 - Clear Cct - Ven D1 | For | X | O | X | O | X | O | O | X | X |
| Return 2 - Clear Cct - Art D1 | Rev | O | X | X | O | X | O | O | X | X |
| Return 1 - Clear Cct - Ven D2 | For | X | O | X | O | X | X | X | O | O |
| Return 2 - Clear Cct - Art D2 | Rev | O | X | X | O | X | X | X | O | O |

In the example illustrations of ultrafiltration operation, blood flow direction is illustrated by the arrows in FIGS. 1-4. The forward direction may refer to a blood flow path from the arterial interface (1) to blood pump (4) to the ultrafiltration device (i.e. one of dialyzer (5) or (6)) and to venous interface (11). The reverse direction may refer to a blood flow path from the venous interface (11) to blood pump (4) to the ultrafiltration device (i.e. one of dialyzer (5) or (6)) and to arterial interface (1).

FIG. 1 illustrates an example blood treatment system (100). In an embodiment, the blood treatment system comprising blood pump (4) for urging blood from an arterial interface (1) or venous interface (1) through a blood flow path (illustrated by arrows); a dialyser (5) or (6) in fluid communication with the blood flow path for ultrafiltering the blood to remove fluid therefrom; a fluid removal pump (16) in fluid communication with the dialyser for urging ultrafiltered fluid away from the dialyser; a controller (21) in signal communication with the blood pump; and a reversing valve (9) for selectively reversing direction of blood flow in at least a portion of the blood flow path under signal control of the controller. As shown in FIGS. 1-4, the portion of the blood flow path in which blood flow is reverse may include arterial interface (1) and venous interface (11). Blood pump (4) may be selectively activatable under signal control of the controller. System (100) may comprise at least one flow control element for directing the blood flow path through a selected one of the first dialyser or the second dialyser under signal control of the controller. Flow control elements may be clamps or valve, e.g. a minimal or zero dead space valve or clamps.

As shown in FIG. 1, blood and fluid are transported between the various elements by tubing segments (2). In Forward Operation D1 or Forward Operation D2 (see Table 1 for valve states), blood pump (4), controlled by controller (21), pumps blood from the arterial access (1). Blood pressure at arterial access (1) is monitored by controller (21) via the arterial pressure sensor (3). Blood then travels through the arterial clamp (22) in the open state. If the pressure is outside of acceptable limits based on the valve states, the controller (21) may trigger a blood access pressure alarm that will stop the blood pump (4). Blood is transported via the blood pump (4) through the reversing valve (9) and then through the first dialyser (5) via the inlet control clamp (25) for dialyzer (5) and the outlet control clamp (26). In some embodiments, reversing value (9) may be a disposable and rotatable blood line reversal valve similar to one described in U.S. Pat. No. 5894011, the entire contents of which are hereby included by reference herein. Alternatively, the blood may be transported via the blood pump (4) through the reversing valve and then through dialyser (6) via the inlet control clamp (27) for dialyzer (6) and the outlet control clamp (27) for dialyzer (6). Reversing valve maybe positioned upstream of an inlet of blood pump (4) for selectively flowing blood from one of the arterial or venous interface to the inlet of the blood pump without reversing the blood pump. After dialyser (5) or dialyser (6), the blood is transported to an air removal system (50) comprising air removal filter (29) where any air bubbles that are present are removed from the blood. In some embodiments, air removal system (50) may be the air removal system described in International Patent Application No. PCT/US2020/20475 (Publication No. WO/2020/176879), the entire contents of which are hereby included by reference herein. Air level in air removal filter (29) may be maintained by the air removal pump (32) in fluid communication with the hydrophobic filter (31) and air removal filter (29). Air removal pump operation may be controlled by control system (33) and/or controller (21) using air level status provided by the air sensor (30). A motor for rotating air removal filter (9) about at least two axis of rotation may be provided to maintain air removal filter (9) in an upright position. Blood may then be transported from air removal filter (29) through an air detector (7). Air detector (7) may be monitored controller (21) which will stop the blood pump and close arterial clamp (22) and venous clamp (23) if air is detected in the blood. If no air is present in the blood, then the blood may be transported through the recirculation valve (8) which is in a closed state, so preventing fluid communication with the blood line connected to the arterial access (1). Blood then travels through reversing valve (9) in the forward mode and then travels through venous clamp (23) in an open state. Pressure of the blood before the venous access (11) may be monitored by controller (21) via the venous pressure access sensor(10). If the venous pressure is outside of acceptable limits based on the valve states, controller (21) will trigger a blood access pressure alarm that will stop the blood pump (4).

In some embodiments, anticoagulant solution present in the anticoagulant reservoir (13) may be pumped by the anticoagulant pump (12) into the blood circuit. The location of the introduction of the anticoagulant solution is shown in FIGS. 1-4 as between the blood pump (4) and the reversing valve (9), but in an alternative implementation, the location of the introduction of the anticoagulant solution may be located between the dialyser (5) or dialyser (6) and the blood pump (4). Examples of anticoagulant solutions may include but are not limited to unfractionated heparin, low-molecular-weight heparin, direct thrombin inhibitors, heparinoids, prostanoids, nafamostat mesilate, citrate infusions.

Fluid removal pump (16) may pump ultrafiltered fluid from the dialyser (5) or dialyser (6) into the fluid removal reservoir (17). Volume of fluid removed and the rate of fluid removed may be controlled by controller (21) by changing the speed that the fluid removal pump (16) runs. Inlet pressure of the fluid removal pump (16) may be monitored by the controller (21) via the ultrafiltered fluid pressure sensor (19). Fluid sensor (20) may be configured to detect blood in the ultrafiltered fluid, and controller (21) may implement logic for disabling blood pump (4) in response to detection of blood by fluid sensor (20).

Transmembrane pressure across the dialyser (5) or dialyser (6) membrane can be calculated by controller (21) as the difference between the pressure reported by venous access pressure sensor (10) when system (100) is the forward mode or the arterial access pressure sensor (3) when system (100) is in the reversed mode, and the pressure reported by the ultrafiltered fluid pressure sensor (19). A signal for a warning alarm may be issued by controller (21) if the pressure reported by the ultrafiltered fluid pressure sensor (19) exceeds a predetermined limit. In example, the predetermined limit of transmembrane pressure may be limit suggestive of fouling and/or damage to the ultrafiltration membrane.

Fluid may be periodically drained from the fluid removal reservoir (17) by opening the fluid disposal control clamp (18). The fluid removal pump (16) may be periodically reversed in a de-fouling cycle in order to clear fouling from the blood surface side of the filter membrane. In an example, pump (16) may urge ultrafiltered fluid into a dialyser to clear fouling from a membrane of the dialyser. Controller (21) may be configured to reverse fluid removal pump (16) in response to determining a transmembrane pressure of a dialyser. The pressure of the fluid at sensor (19) may be monitored by controller (21) in order to adjust the flow rate of the fluid removal pump in order to maximize the removal of fouling from the blood membrane of the dialyser (5) or dialyser (6). The volume of fluid returned to the blood system by the fluid removal pump (16) may be calculated by controller (21) using the pump rate and duration so as to increase the rate of the fluid removal pump (16) when the system returns to normal ultrafiltration mode to compensate for the fluid that was returned in the de-fouling cycle.

System (100) may also comprise a prime fluid reservoir (14) to contain fluid for priming and/or flushing system (100). System (100) may also comprises a plurality of valves, e.g. clamps (15), (22)-(28), recirculation valve (8), and reversing valve (9), each under signal control of controller (21) to effect automated priming of system (100) by circulating prime fluid urged by blood pump (4) from prime fluid reservoir (14) along selected fluid flow paths. In an embodiment, the selected fluid flow paths includes a fluid flow path connecting the prime fluid reservoir (14) and dialyser (5). In another example, the selected fluid flow paths includes a fluid flow path connecting the prime fluid reservoir (14) and dialyser (6). In another example, the selected fluid flow paths includes a fluid flow path connecting prime fluid reservoir (14) and arterial interface (1). In another example, the selected fluid flow paths includes a fluid flow path connecting prime fluid reservoir (14) and venous interface (11). System (100) may also comprise controller (21) which is configured to control the valves of system (100) to remove dialyser (5) or (6) from the blood flow path to facilitate replacement of the dialyser.

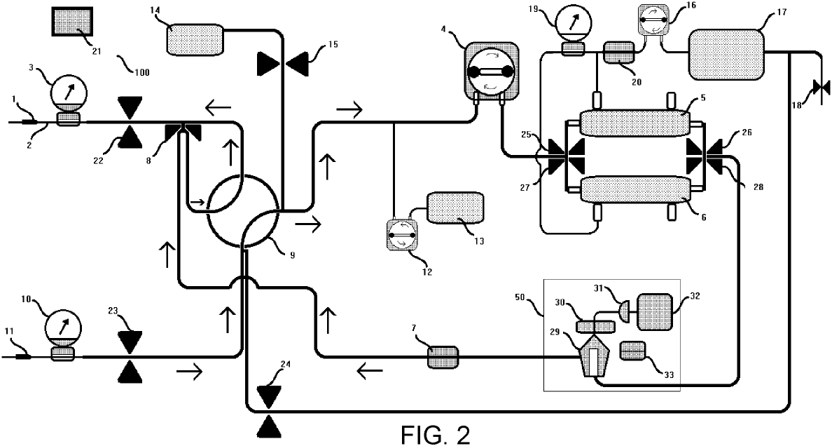

FIG. 2 illustrates example blood treatment system (100) in reverse operation. In Reverse Operation D1 or Reverse Operation D2 (see Table 1 for example valve states), blood pump (4), controlled by controller (21), pumps blood from venous access (11). Blood pressure at the venous access (11) is monitored by controller (21) via the venous pressure sensor (18). Blood may then travels through the venous clamp (23) in the open state. If the pressure is outside of acceptable limits based on the valve states, controller (21) will may signal a blood access pressure alarm that will stop the blood pump (4). Blood is transported via the blood pump (4) through reversing valve (9) and then through the first dialyser (5) via the inlet control clamp (25) of dialyser (5) and the outlet control clamp (26) for dialyser (5). Alternatively, blood may be transported via the blood pump (4) through reversing valve (9) and then through the second dialyser (6) via the inlet control clamp (27) for dialyser (6) and the outlet control clamp (27) of dialyzer (6). After the dialyser (5) or dialyser (6), the blood is transported to the air removal system (5), comprising air removal filter (29), where any air bubbles that are present are removed from the blood. Air level in the air removal filter (29) may be maintained by the air removal pump (32) in fluid communication with the hydrophobic filter (31) and air removal filter (29). Air removal pump operation may be controlled microprocessor control system (33) and/or controller (21) using air level status provided by the air sensor (30). Blood may then be transported from the air removal filter (29) through an air detector (7). Air detector (7) is monitored by controller (21) which may stop blood pump (4) and close the arterial clamp (22) and the venous clamp (23) if air is detected in the blood. If no air is present in the blood, then the blood is transported through the recirculation valve (8) which is in a closed state, so preventing fluid communication with the blood line connected to the arterial access (1). The blood then travels through the reversing valve (9) in the reverse mode and to arterial clamp (22) in an open state. Blood pressure before the arterial access (1) is monitored by the controller (21) via the arterial pressure access sensor (3). If the pressure at arterial pressure access sensor (3) is outside of acceptable limits based on the valve states, controller (21) may signal a blood access pressure alarm and stop the blood pump (4).

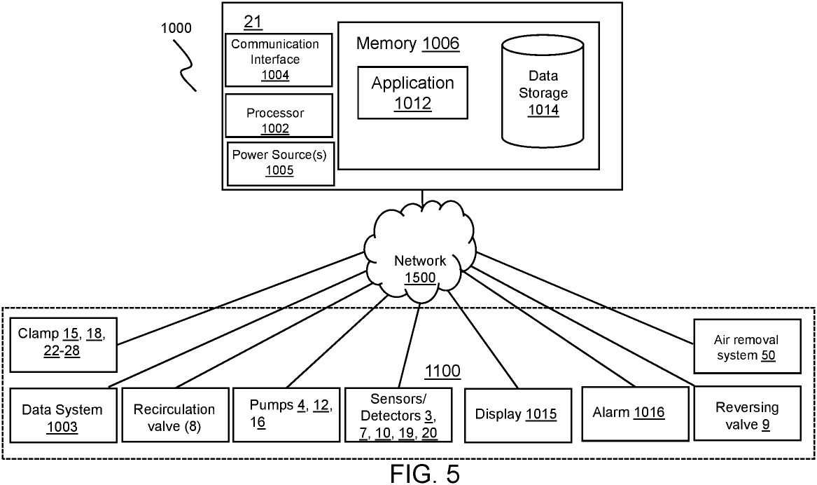

FIG. 5 shows an example system (1000) for controlling a blood treatment system. System (1000) may comprise controller (21), described herein. Controller (21) includes a processor (1002) configured to implement processor readable instructions that, when executed, configure the processor (1002) to conduct operations described herein. The processor (1002) may be a microprocessor or microcontroller, a digital signal processing (DSP) processor, an integrated circuit, a field programmable gate array (FPGA), a reconfigurable processor, a programmable read-only memory (PROM), or combinations thereof. In a non-limiting example micro-controller may be provided by Microchip Technology Part # PIC16F18326T-I/JQ and supplied by Digikey Part # PIC16F18326T-I/JQCT-ND. Controller (21) may be coupled to a plurality of power sources 1005, e.g. battery input, to provide a portable power supply. Controller (21) may include a communication interface (1004) to communicate with other computing or sensor devices, to access or connect to network resources, or to perform other computing applications by connecting to a network (or multiple networks) capable of carrying data. In some examples, the communication interface (1004) may include one or more busses, interconnects, wires, circuits, and/or any other connection and/or control circuit, or combination thereof. The communication interface (1004) may provide an interface for communicating data between the system (1000) and a display (1015) or an alarm (1016). An alarm described herein, may be any indication provided to a user of the wearable renal therapy device that corrective action should be taken. Non-limiting examples of alarms are visual alerts on a display or a light of the wearable device; vibrations from a vibration actuator of the wearable device; or auditory alerts from a speaker of the wearable device.

Controller (21) may also comprise connections for communicating with any pump of a renal therapy system according to this disclosure. Controller (21) may comprise a sensor connection for monitoring a pump rotation status, e.g. of blood pump (4). In an embodiment, the sensor connection may be an encoder connection.

Controller (21) may comprise a connection(s) to a pressure sensor, e.g. pressure sensors (3) (10) on the arterial and venous lines respectively, and/or a second connection (910). Controller (21) may also comprise a connection to fluid detector (20) for detecting blood in ultrafiltrated fluid, a connection for the air detector (7) and a connection for an optional sensor(s) to reservoirs (13), (14), (17) to indicate if the reservoir(s) are empty and/or full. Controller may also comprise additional sensor and driver ports to enable additional treatment modalities.

Controller (21) may be coupled to a data system (1003) for storing treatment data of a blood treatment device (e.g. an SD Card port and SD Card, digital data storage memory card, TF card, USB flash drive storage element, and/or may be configured to communicate with cloud services such as iCloud, Dropbox, Google clouds, or any other digital data servers). Data system (1003) may also comprises a universal asynchronous receiver-transmitter (UART) to allow communication with other devices, e.g. a smartphone or a computer, for transmitting data for analysis and/or storage. UART may include or be coupled to a wireless transceiver for wireless communication with such other devices, e.g., by way of infra-red, Bluetooth, Wi-Fi, or the like. Controller (21) may also be coupled to clamps (15), (18), (22)-(28), recirculation valve (8), reversing valve (9), blood pump (4), anticoagulant pump (12), fluid removal pump (16), air removal system (50), arterial access pressure sensor (3), venous access pressure sensor (10), ultrafiltered fluid pressure sensor (19), fluid detector (20), air detector (7), display (1015), and/or alarm (1016) via a network (1500). Network (1500) may include any wired or wireless communication path, such as an electrical circuit. In some embodiments, the network (1500) may include one or more busses, interconnects, wires, circuits, and/or any other connection and/or control circuit, or a combination thereof. In some embodiments, the network (1500) may include a wired or a wireless wide area network (WAN), local area network (LAN), a combination thereof, or the like. In some embodiments, the network (1500) may include a Bluetooth® network, a Bluetooth® low energy network, a short-range communication network, or the like.

In some embodiments, controller (21) may generate a data signal encoding an alarm condition and transmit the data signal to another device (e.g., a smartphone or computer) to present the alarm condition to the patient. The data signal encoding the alarm condition may be transmitted to such other device (or devices) in real-time or near real-time. In some embodiments, the alarm condition may be encoded for display at a display (1015).

Controller (900) may include memory (1006). The memory (1006) may include one or a combination of computer memory, such as static random-access memory (SRAM), random-access memory (RAM), read-only memory (ROM), electro-optical memory, magnetooptical memory, erasable programmable read-only memory (EPROM), and electrically-erasable programmable read-only memory (EEPROM), Ferroelectric RAM (FRAM) or the like.

The memory (1006) may store an application (1012) including processor readable instructions for conducting operations described herein. In some examples, the application (1012) may include operations for controlling a blood treatment system. The method comprises: causing blood pump (4) to flow blood through a flow path from one of an arterial interface (1) or venous interface (11) to a dialyser (5) or (6) and to the other of the arterial or venous interface; and causing reversing valve (9) to reverse the flow of blood through a portion of the flow path. In an embodiment, causing the reversing valve to reverse the flow of blood through the portion of the flow path comprises causing blood to reverse flow between the arterial interface (1) and venous interface (11). In another embodiment, reversing valve (9) is positioned upstream of an inlet of blood pump (4), and the method comprises selectively flowing blood from one of the arterial or venous interface to the inlet of the blood pump without reversing the blood pump to mitigate against the risk of air embolism or undetected needle separation.

In another example, application (1012) may include operations to cause system (100) to treat blood. In the example, fluid removal pump (16) runs in a forward direction at a rate controlled by controller (21) in order to remove fluid from the blood being pumped through the dialyser (5) or (6). The removed fluid is continually monitored for the presence of blood by the blood in fluid detector (20). If blood is sensed in the dialyser (5) or (6), the fluid removal pump (16) is stopped, an alarm is initiated by controller (21) and the patient is prompted to initiate an automated dialyser change operation. Pressure of the fluid being removed from the dialyser is continually measured by the ultrafiltered fluid pressure sensor (19). Transmembrane pressure across the dialyser is calculated by controller (21) by comparison of the pressure indicated by the ultrafiltered fluid pressure sensor (19) with the pressure reading indicated by the arterial access pressure sensor (3). An algorithm in controller (21) may determines if the change in the transmembrane pressure indicates possible fouling and initiates the fouling removal process if needed. If the fouling removal process did not adequately reduce the transmembrane pressure, the controller (21) may initiates a TMP alarm and the patient is prompted to initiate an automated dialyser change operation.

In another example, application (1012) may include operations to signal an alarm when a sensor indicates that hydrophobic filter (31) is full; anticoagulant reservoir (13) is empty; fluid reservoir (17) is full; priming solution container (14) is empty; air detector (7) detects bubbles of air; blood is detected at fluid detector (20), and/or a threshold pressure (e.g. low pressure value or high pressure value) is measured at any one of pumps (4), (12), or pressure sensors (3), (10), (19). Non-limiting examples of alarms are visual alerts on a display or a light on the renal therapy device; vibrations from a vibration actuator of the renal therapy device; or auditory alerts from a speaker of the renal therapy device.

In another example, application (1012) may include operations to pump anticoagulant solution into blood within system (100). Anticoagulant solution present in the anticoagulant reservoir (13) may be pumped by the anticoagulant pump (12) into the blood circuit. The location of the introduction of the anticoagulant solution is shown in FIGS. 1-4 as between the blood pump (4) and the reversing valve (9), but in alternative implementations, the location of the introduction of the anticoagulant solution may be located between the dialyser (5) or dialyser (6) and the blood pump (4).

In another example, application (1012) may include operations to pump ultrafiltrated fluid from dialyzer (5) or (6). Fluid removal pump (16) may pump ultrafiltered fluid from the dialyser (5) or dialyser (6) into the fluid removal reservoir (17). Volume of fluid removed and the rate of fluid removed may be controlled by controller (21) by changing the speed that the fluid removal pump (16) runs. Inlet pressure of the fluid removal pump (16) is monitored by the microprocessor control system (21) via the ultrafiltered fluid pressure sensor (19). Transmembrane pressure across the dialyser (5) or dialyser (6) membrane can be calculated by controller (21) as the difference between the pressure reported by the venous access pressure sensor (10) when in the forward mode or the arterial access pressure sensor (3) when in the reversed mode, and the pressure reported by the ultrafiltered fluid pressure sensor (19). A warning alarm may be issued by controller (21) if this exceeds a predetermined limit. Fluid may be periodically drained from the fluid removal reservoir (18) by opening the fluid disposal control clamp (18).

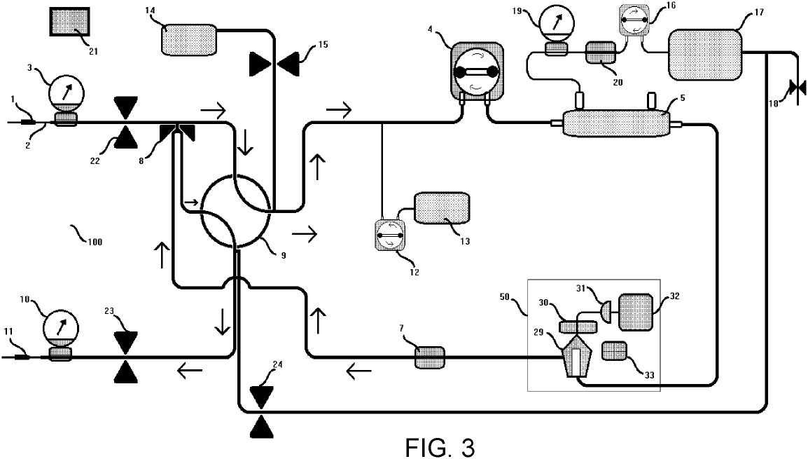

In another example, application (1012) may include operations to prime system (100) to remove entrained air. The operations may cause a plurality of flow valves to effect automated priming of a portion of the flow path by circulating prime fluid urged by the blood pump from a prime fluid reservoir along the portion of the flow path. The operations may be automated and controlled by controller (21) In an example priming method, the valves of system (100) may be initially in the following state (see Table 1 Prime 1 - Intake - D1): the reversing valve (9) is in the forward position, as shown in FIGS. 1 and 3, arterial clamp (22) is in the closed position, the venous clamp (23) is in the closed position, the recirculation valve (8) is in the closed position, the prime fluid control clamp (15) is in the open position, the flush fluid control clamp (24) is in the open position, the inlet control clamp (25) for dialyzer (5) is in the open position, the outlet control clamp (26) for dialyzer (6) is in the open position, the inlet control clamp (27) for dialyser (5) is in the closed position and the outlet control clamp (28) for dialyzer (6) is in the closed position. During the example priming process, automatic air removal system (50) may remove most of the accumulated air in the system. A small amount of accumulated air may be directed to the fluid removal reservoir (17).

Continuing the example priming method, blood pump (4) may start and draw fluid from prime fluid reservoir (14) for a specified period of time. Priming fluid may travel through the fluid path based on the position of the clamps and through dialyser (5) to eventually go into the fluid removal reservoir (17). Blood pump (4) may then stop. Inlet control clamp (25) may then switches from the open position to the closed position, the outlet control clamp (26) switches from the open position to the closed position to isolate dialyzer (5) from the flow path; and the inlet control clamp (27) switches from the closed position to the open position and the outlet control clamp (28) switches from the closed position to the open position such that dialyzer (6) joins the flow path (see Table 1 Prime 1 - Intake D2). Blood pump (4) may then starts and draws fluid from prime fluid reservoir (14) for a specified period of time. This fluid travels through the fluid path based on the position of the clamps and through dialyser (6) to eventually go into the fluid removal reservoir (17). Blood pump (4) may then stop.

Continuing the example, priming fluid may be recirculated in system (100). Recirculation valve (8) may open and the flush fluid control clamp (24) close (see Table Prime 2 - Circulate - D2). Blood pump (4) may start and circulate the priming fluid in the circuit for a specified period of time. Fluid removal pump (16) may runs for a specified period of time to prime the fluid space of dialyser (6) and fluid detector (20). Blood pump (4) may then stop. Inlet control clamp (25) may then switch from the closed position to the open position, the outlet control clamp (26) switches from the closed position to the open position such that dialyzer (5) joins the flow path; and inlet control clamp (27) switches from the open position to the closed position and the outlet control clamp (28) switches from the open position to the closed position to isolate dialyzer (6) (see Table 1 Prime 2 - Circulate - D1). Blood pump (4) may start and circulate the fluid in the circuit for a specified period of time. Fluid removal pump (16) may runs for a specified period of time to prime the fluid space of dialyser (5), the ultrafiltered fluid pressure sensor (19) and fluid detector (20). Blood pump (4) may then stop.

Continuing the example, recirculation valve (8) may then close and flush fluid control clamp (24) opens (See Table 1 Prime 3 - Flush Circuit - D1). Blood pump (4) may start and draw fluid from prime fluid reservoir (14) for a specified period of time. This fluid may travels through the fluid path based on the position of the clamps and through dialyser (5) to eventually go into the fluid removal reservoir (17). Blood pump (4) may then stop. Inlet control clamp (25) may then switch from the open position to the closed position, the outlet control clamp (26) switches from the open position to the closed position, the inlet control clamp (27) switches from the closed position to the open position and the outlet control clamp (28) switches from the closed position to the open position (See Table 1 Prime 3 - Flush Circuit -D2). Blood pump (4) may then start and draw fluid from prime fluid reservoir (14) for a specified period of time. This fluid travels through the fluid path based on the position of the clamps and through dialyser (6) to eventually go into the fluid removal reservoir (17). The blood pump (4) may then stop.

To prime venous access (11), a patient may then uncap the venous access (11) for priming, e.g. by holding venous access (11) over a container to drain or connecting a disposable collection bag to the venous access (11). Venous clamp (23) switches from closed to open, and the flush fluid control clamp (24) switches from open to closed (see Table 1 Prime 3 - Flush Ven - D2). Blood pump (4) may start and draw fluid from prime fluid reservoir (14) for a specified period of time. Fluid travels through the fluid path based on the position of the clamps and through dialyser (6) to eventually exit the system at the venous access (11). Blood pump (4) may then stops and the patient may disconnect the disposable collection bag (if used) and recap the venous access (11).

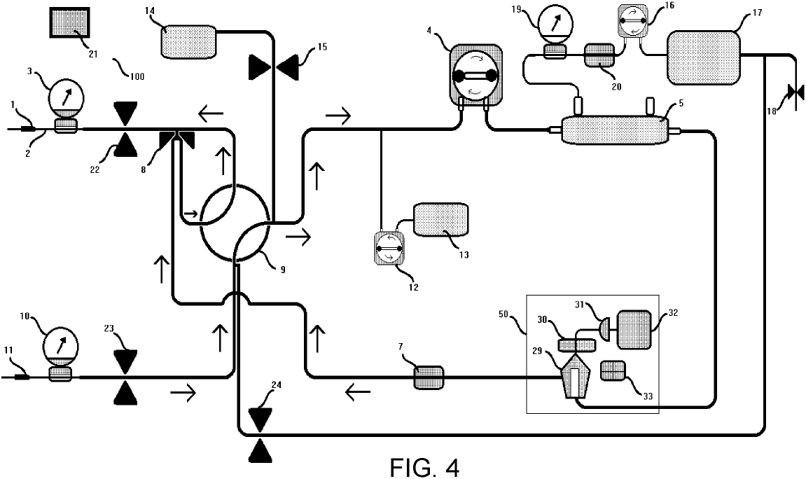

To prime arterial access (1), the patient may uncap the arterial access (1) for priming, e.g. by holding arterial access (1) over a container to drain or connecting a disposable collection bag to the arterial access (1). Reversing valve (9) may switches from forward to reverse as shown in FIGS. 2 and 4, and the arterial clamp (22) switches from closed to open, and the venous clamp (23) switches from open to closed. Inlet control clamp dialyser one (25) may then switch from the closed position to the open position, outlet control clamp (26) switches from the closed position to the open position, inlet control clamp (27) switches from the open position to the closed position and the outlet control clamp (28) switches from the open position to the closed position (see Table 1 Prime 3 - Flush Art - D1). Blood pump (4) may start and draws fluid from prime fluid reservoir (14) for a specified period of time. This fluid travels through the fluid path based on the position of the clamps and through dialyser (5) to eventually exit the system at the arterial access (1). Blood pump (4) may then stop and the patient may disconnect the disposable collection bag (if used) and recap the arterial access (1).

As shown in FIGS. 3 and 4, system (100) according to this disclosure may have a single dialyzer. Example methods effecting priming and the blood return described herein may be applied to single dialyzer systems by skipping dialyser switching steps. FIG. 3 is an example of single dialyzer system in the forward mode. FIG. 4 is an example of single dialyzer system in the reverse mode.

In another example, application (1012) may include operations to disconnect system (100) from a patient. System (100) may be disconnected from a patient in an example method that is automated and controlled by controller (21). In an example, the valves of system (100) are initially in the following state: the reversing valve (9) is in the forward position as in FIGS. 1 and 3, arterial clamp (22) is in the closed position, venous clamp (23) is in the open position, recirculation valve (8) is in the closed position, prime fluid control clamp (15) is in the open position, flush fluid control clamp (24) is in the closed position, inlet control clamp dialyser (25) is in its existing state, the outlet control clamp dialyser (26) is its existing state, the inlet control clamp dialyser (27) is its existing state and the outlet control clamp dialyser (28) is its existing state. During this entire process, the automatic air removal system (50) may remove most of the accumulated air in the system. Blood pump (4) may start and draw fluid from prime fluid reservoir (14) for a specified period of time. This fluid travels through the fluid path based on the position of the clamps and through to eventually exit the system at the venous access (11). Blood pump (4) may then stop. Reversing valve (9) switches to the reverse position as in FIGS. 2 and 4 and arterial clamp (22) switches to the open position and the venous clamp (24) switches to the closed position. Blood pump (4) may then start and draw fluid from prime fluid reservoir (14) for a specified period of time. This fluid travels through the fluid path based on the position of the clamps and through to eventually exit the system at the arterial access (1). The patient may then physically disconnect the access lines at interface (1) and (11).

In another example, application (1012) may include operations to perform a fouling removal method which may be automated by controller 21. Controller (21) may signal fluid removal pump (16) to run backwards to pump fluid from the fluid removal reservoir (17) into the dialyser membrane in use at a rate sufficient to clear accumulated fouling from the blood side of the membrane in use. The pressure of the fluid is monitored by the ultrafiltered fluid pressure sensor (19). If a predetermined pressure determined by the algorithm of the controller (21) is exceeded, the fouling removal method is stopped and the patient is prompted to initiate a dialyser change operation. The controller (21) may track of the volume of fluid returned to the blood by counting the revolutions of the fluid removal pump. The volume of fluid returned to the blood may be added to the fluid removal target for the treatment. Once an allotted time for fouling removal has occurred as determined by controller (21) the fluid removal pump (16) runs forward again to resume ultrafiltration.

In another example, application (1012) may include operations to switch between two dialyzers. System (100) may perform a process of switching fluid flow from dialyser (5) to dialyser (6). In the example, when controller (21) determines that the dialyser needs to be switched, blood pump (4) may stop, reversing valve may switch to forward mode as in FIG. 1, prime fluid control clamp (15) may open, and arterial clamp (22) will close (see Table 1 - Clear Cct - Ven D1). Blood pump my then start and run a predetermined length of time to clear the blood from dialyser (5). Blood pump (4) may then stop. Prime fluid control clamp (15) closes, arterial clamp (22) opens, inlet control clamp (25) closes, outlet control clamp (26) closes, inlet control clamp (27) opens and the outlet control clamp (28) opens. Blood pump (4) may then start such that system (100) runs on dialyser (6). In another example, switching from dialyser (6) to (5) may be achieved following the same steps noted above except dialyzer (6) may be cleared with the valves in the positions indicated in Table 1 Return 1 - Clear Cct - Vent D2, and once completed inlet control clamp (25) opens, outlet control clamp (26) opens, inlet control clamp (27) closes and the outlet control clamp (28) closes. Blood pump (4) may then start such that system (100) runs on dialyser (5). Dialyzer (5) or (6) may be cleared through the arterial interface (1) using the valve settings illustrated in Table 1 Return 2 - Clear Cct - Art D1; and Table 1 Return 2 - Clear Cct - Art D2, respectively.

In another example, application (1012) may include operations to replace dialyser (5) or (6) and switch to it. If both dialysers have been used and a patient decides that only one additional dialyser is required to complete the treatment, then method may be followed. In this example, system (100) is running on dialyser (6). Fluid removal pump (16) stops and blood pump (4) stops. Prime fluid control clamp (15) opens, and the arterial clamp (22) closes. Blood pump (4) starts and runs for a predetermined period of time to clear the blood from dialyser (6) and the blood circuit through to the venous access (11). Blood pump (4) stops. Reversing valve (9) goes to reverse mode as in FIG. 2, and venous clamp (23) closes. Blood pump starts and runs a predetermined period of time so as to clear the blood from the arterial access. Blood pump (4) stops. Flush fluid control clamp (24) opens, the arterial clamp (22) closes, the inlet control clamp (25) for dialyzer (5) closes, the outlet control clamp (26) closes, the inlet control clamp (27) for dialyser (6) closes, and the outlet control clamp (28) closes. The patient may be is prompted by an alarm signaled by controller (21) to replace dialyser one (5) and the patient may confirm to controller (21) using communication interface (1004) when it is complete. The inlet control clamp (25) for dialyzer (5) opens, and the outlet control clamp (26) opens. Blood pump (4) starts and runs a predetermined period of time. Controller (21) may add the volume of fluid used to the fluid removal target. Blood pump (4) stops. Prime fluid control clamp (15) closes, the reversing valve (9) goes to forward mode as in FIG. 1, the flush fluid control clamp (24) closes, arterial clamp (22) opens, the venous clamp opens. Blood pump (4) starts and system (100) is now running on dialyser (5) and fluid removal may resume.

In another example, application (1012) may include operations to replace two dialysers. If both dialysers have been used and the patient decides that at least two additional dialysers are required to complete the treatment, then this example method may be followed. It is assumed that the system is running on dialyser (6). The fluid removal pump (16) stops and blood pump (4) stops. The prime fluid control clamp (15) opens, and the arterial clamp (22) closes. The blood pump (4) starts and runs for a predetermined period of time to clear the blood from dialyser (6) and the blood circuit through to the venous access (11). The blood pump (4) stops. The reversing valve (9) goes to reverse mode as in FIG. 2, the venous clamp (23) closes. The blood pump starts and runs a predetermined period of time so as to clear the blood from the arterial access (1). The blood pump (4) stops. The flush fluid control clamp (24) opens, arterial clamp (22) closes, the inlet control clamp (25) for dialyzer (5) closes, the outlet control clamp (26) closes, the inlet control clamp (27) for dialyzer (6) closes, the outlet control clamp (28) also closes. The patient may be prompted an alarm signed by controller (21) to replace dialyser (5) and to replace dialyser (6), the patient confirms to controller (21) when it is complete. The inlet control clamp (27) for dialyzer (6) opens, and the outlet control clamp (28) opens. Blood pump (4) starts and runs a predetermined period of time. Controller (21) adds the volume of fluid used to the fluid removal target. The inlet control clamp (27) for dialyzer (6) closes, the outlet control clamp (28) closes, the inlet control clamp (25) for dialyzer (5) opens, the outlet control clamp (26) also opens. Blood pump (4) starts and runs a predetermined period of time. Controller (21) adds the volume of fluid used to the fluid removal target. Blood pump (4) stops. Prime fluid control clamp (15) closes, reversing valve (9) goes to forward mode as in FIG. 1, the flush fluid control clamp (24) closes, arterial clamp (22) opens, the venous clamp opens. Blood pump (4) starts and the system is now running on dialyser (5) and fluid removal resumes.

The valve and clamps may be used interchangeable. Each valve and/or clamp according to this disclosure may be a minimal dead space or zero dead space. For example, recirculating valve and/or clamps of this disclosure may have minimal dead space, where dead space means a portion of the flow path where blood may be stopped from flowing for a period of time longer than 30 seconds. A minimal dead space valve may be, but is not limited to being a pinch valve that occludes tubing. Other options for this valve design may be, but are not limited to are a rotating valve, a diaphragm valve operated by an electric solenoid, a diaphragm valve operated by an air pressure, a diaphragm valve operated by an air vacuum, a diaphragm valve operated by a fluid pressure, diaphragm valve operated by a cam actuator.

Although terms such as “maximize”, “minimize” and “optimize” may be used in the present disclosure, it should be understood that such term may be used to refer to improvements, tuning and refinements which may not be strictly limited to maximal, minimal or optimal.

The term “connected” or “coupled to” may include both direct coupling (in which two elements that are coupled to each other contact each other) and indirect coupling (in which at least one additional element is located between the two elements).

The term “isolate” refers to removing devices (e.g. a dialyzer) from a flow path of fluid (e.g. blood or priming fluid) through the systems described herein.

The term “substantially” as used herein may be applied to modify any quantitative representation which could permissibly vary without resulting in a change in the basic function to which it is related.

The above description is meant to be exemplary only, and one skilled in the relevant arts will recognize that changes may be made to the embodiments described without departing from the scope of the invention disclosed. The present disclosure may be embodied in other specific forms without departing from the subject matter of the claims. The present disclosure is intended to cover and embrace all suitable changes in technology. Modifications which fall within the scope of the present invention will be apparent to those skilled in the art, in light of a review of this disclosure, and such modifications are intended to fall within the appended claims. Also, the scope of the claims should not be limited by the preferred embodiments set forth in the examples, but should be given the broadest interpretation consistent with the description as a whole.

As one of ordinary skill in the art will readily appreciate from the disclosure, processes, machines, manufacture, compositions of matter, means, methods, or steps, presently existing or later to be developed, that perform substantially the same function or achieve substantially the same result as the corresponding embodiments described herein may be utilized. Accordingly, the appended claims are intended to include within their scope such processes, machines, manufacture, compositions of matter, means, methods, or steps.

The description provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements Band D, then the inventive subject matter is also considered to include other remaining combinations of A, B, C, or D, even if not explicitly disclosed.

The embodiments of the devices, systems and methods described herein may be implemented in a combination of both hardware and software. These embodiments may be implemented on programmable computers, each computer including at least one processor, a data storage system (including volatile memory or non-volatile memory or other data storage elements or a combination thereof), and at least one communication interface.

Program code is applied to input data to perform the functions described herein and to generate output information. The output information is applied to one or more output devices. In some embodiments, the communication interface may be a network communication interface. In embodiments in which elements may be combined, the communication interface may be a software communication interface, such as those for interprocess communication. In still other embodiments, there may be a combination of communication interfaces implemented as hardware, software, and combination thereof.

Throughout the foregoing discussion, numerous references will be made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements.

As can be understood, the examples described above and illustrated are intended to be exemplary only.

1. A blood treatment system comprising:
a blood pump for urging blood from an arterial or venous interface through a blood flow path;
a dialyser in fluid communication with said blood flow path for ultrafiltering the blood to remove fluid therefrom;
a fluid removal pump in fluid communication with said dialyser for urging ultrafiltered fluid away from said dialyser;
a controller in signal communication with said blood pump; and
a reversing valve for selectively reversing direction of blood flow in at least a portion of the blood flow path under signal control of said controller,
wherein said blood pump is selectively activatable under signal control of said controller; and
wherein said system is portable and wearable by a patient undergoing blood treatment.

2. The system of claim 1, wherein said dialyser is a first dialyser and said system further comprises a second dialyser; and wherein the system comprises at least one flow control element for directing said blood flow path through a selected one of said first dialyser or said second dialyser under signal control of said controller.

3. (canceled)

4. (canceled)

5. The system of claim 1, where the portion of the blood flow path comprises the arterial and the venous interface.

6. The system of claim 1, wherein the reversing valve is positioned upstream of an inlet of the blood pump for selectively flowing blood from one of the arterial or venous interface to the inlet of the blood pump without reversing the blood pump.

7. The system of claim 1, comprising a prime fluid reservoir; and a plurality of other valves, each under signal control of said controller to effect automated priming of the system by circulating prime fluid urged by said blood pump from said prime fluid reservoir along selected fluid flow paths.

8. (canceled)

9. (canceled)

10. (canceled)

11. The system of claim 7, wherein said selected fluid flow paths includes at least one of: a fluid flow path connecting said prime fluid reservoir and said first dialyser; a fluid flow path connecting said prime fluid reservoir and said second dialyser; a fluid flow path connecting said prime fluid reservoir and said arterial interface; and/or a fluid flow path connecting said prime fluid reservoir and said venous interface.

12. (canceled)

13. (canceled)

14. (canceled)

15. The system of claim 7, wherein said controller is configured to control said other valves to remove said first dialyser from said blood flow path to facilitate replacement of said first dialyser; or said controller is configured to control said other valves to remove said second dialyser from said blood flow path to facilitate replacement of said second dialyser.

16. (canceled)

17. (canceled)

18. (canceled)

19. (canceled)

20. (canceled)

21. (canceled)

22. The system of claim 1, comprising an anticoagulant source in fluid communication with the blood flow path, for adding anticoagulant to the blood flow path.

23. The system of claim 1, comprising a pressure sensor for measuring pressure of the blood proximate said arterial or venous interface; wherein said controller implements logic for disabling said blood pump in response to detection of the pressure of the blood by said pressure sensor in excess of a pre-defined limit.

24. (canceled)

25. The system of claim 1, comprising a blood sensor for sensing blood in the ultrafiltered fluid; wherein said controller implements logic for disabling said blood pump in response to detection of blood by said blood sensor.

26. (canceled)

27. The system of claim 1, wherein said fluid removal pump is reversible under signal control of said controller, to urge ultrafiltered fluid into the dialyser to clear fouling from a membrane of the dialyser.

28. The system of claim 1, wherein said controller is configured for reversing said fluid removal pump in response to determining a transmembrane pressure of said dialyser.

29. The system of claim 1, wherein said controller is configured for controlling a pump rate of said blood pump based on a measured fluid removal rate.

30. The system of claim 1, further comprising non-transitory memory for storing data received at said controller; wherein said data includes sensor data.

31. (canceled)

32. (canceled)

33. (canceled)

34. A method for controlling a blood treatment system, the method comprising:
causing a blood pump to flow blood through a flow path from one of an arterial or venous interface to a dialyser and to the other of the arterial or venous interface; and
causing a reversing valve to reverse the flow of blood through a portion of the flow path.

35. The method of claim 34, wherein causing the reversing valve to reverse the flow of blood through the portion of the flow path comprises causing blood to reverse flow between the arterial and venous interface.

36. The method of claim 34, wherein the reversing valve is positioned upstream of an inlet of the blood pump, and the method comprises selectively flowing blood from one of the arterial or venous interface to the inlet of the blood pump without reversing the blood pump.

37. The method of claim 34, comprising directing said blood flow path through a selected one of a first dialyser or a second dialyser.

38. The method of claim 34, comprising causing a plurality of flow valves to prime a portion of the flow path by circulating prime fluid urged by said blood pump from a prime fluid reservoir along the portion of the flow path; wherein said portion of the flow path is isolated from a second dialyser.

39. (canceled)

40. (canceled)

41. The method of claim 38, wherein said portion of the flow path includes said prime fluid reservoir and said arterial interface.
